(19) United States
(12) Patent Application Publication
(10) Pub. No.: US 2023/0212108 A1
(43) Pub. Date: Jul. 6, 2023

(54) LINEAR CARBONATE AND PREPARATION METHOD THEREOF

(71) Applicant: SHENZHEN CAPCHEM TECHNOLOGY CO., LTD., SHENZHEN (CN)

(72) Inventors: YUANYUAN KANG, SHENZHEN (CN); PENGCHENG TU, SHENZHEN (CN); XIANSHUAI ZOU, SHENZHEN (CN); XIWU TANG, SHENZHEN (CN); SENWEI CHEN, SHENZHEN (CN)

(21) Appl. No.: 18/080,682

(22) Filed: Dec. 13, 2022

Related U.S. Application Data
(63) Continuation of application No. PCT/CN2022/123773, filed on Oct. 8, 2022.

(30) Foreign Application Priority Data
Dec. 31, 2021 (CN) 202111670151.4

(51) Int. Cl. C07C 69/96

(52) U.S. Cl. CPC C07C 69/96

(57) ABSTRACT

The present invention relates to the technical field of chemical engineering, and in particular to a linear carbonate and a preparation method thereof, which includes one or more of compounds of structural formula 1 below:

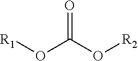

wherein R₁ and R₂ are respectively selected from one of alkyl groups containing 1˜4 carbon atoms;

a hydroxyl concentration of the linear carbonate is no more than 100 ppm, and a free acid conversion rate of a solution with a concentration of 1 mol/L as formulated from the linear carbonate and lithium hexafluorophosphate is less than 1.2 after storage under 25° C. for 30 days. An acidity conversion rate was reduced when lithium hexafluorophosphate is dissolved in the linear carbonate by controlling the hydroxyl concentration, the energy density, discharge capacity, safety performance and service life of a battery can be improved when it's electrolyte solution contains the linear carbonate.

### TECHNICAL FIELD

The present invention relates to the technical field of chemical engineering, and in particular to a linear carbonate and a preparation method thereof.

### BACKGROUND

A linear carbonate is an excellent solvent for an electrolyte solution of a lithium-ion battery because of its low viscosity, high dielectric constant and strong solubility for a lithium salt. It can improve the energy density and discharge capacity of the battery, and further improve the safety performance and service life of the battery. Therefore, it is urgent to develop a linear carbonate with a more stable performance.

### SUMMARY

In order to solve the aforementioned technical problem, the present invention provides a linear carbonate and a preparation method thereof. This method reduces an acidity conversion rate after lithium hexafluorophosphate is dissolved in the linear carbonate by controlling the hydroxyl concentration in the linear carbonate, so that a formulated electrolyte solution of a lithium battery can avoid the influence on the performance of the battery due to the increase in acidity.

The present invention provides a linear carbonate, which includes one or more of compounds of structural formula 1 below:

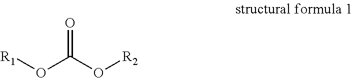

wherein R1 and R2 are respectively selected from one of alkyl groups containing 1˜4 carbon atoms; a hydroxyl concentration of the linear carbonate is no more than 100 ppm, and a free acid conversion rate of a solution with a concentration of 1 mol/L as formulated from the linear carbonate and lithium hexafluorophosphate is less than 1.2 after sealed storage under a condition of a constant temperature of 25° C. for 30 days.

In some embodiments of the present invention, the compound of structural formula 1 is selected from one or more of the following compounds:

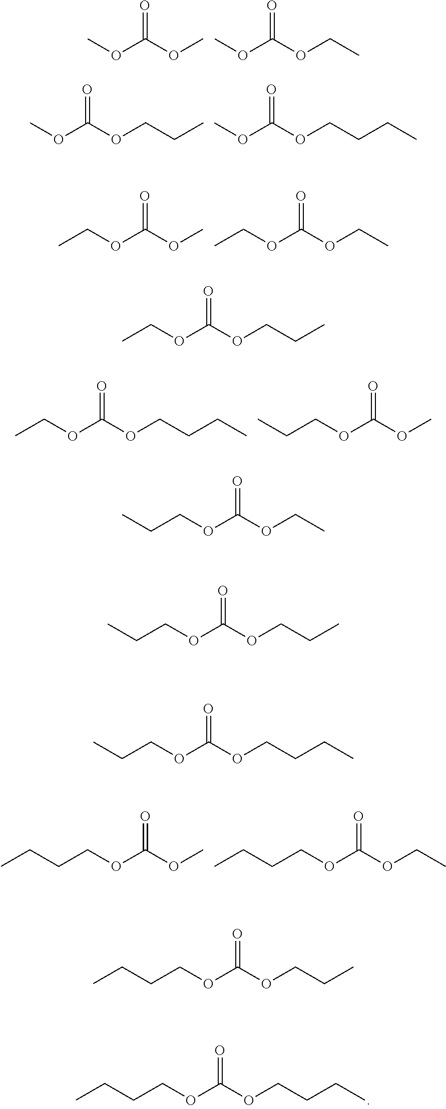

In some embodiments of the present invention, the hydroxyl concentration of the linear carbonate is no more than 60 ppm.

In a second aspect, the present invention provides a method for preparing the aforementioned linear carbonate, which includes the following preparation steps:

step 1: mixing ethylene carbonate with a catalyst and then adding the mixture into a reaction rectification tower, and introducing an alcohol compound for transesterification;

step 2: introducing a condensed fraction collected at a top of the reaction rectification tower into a refining tower, controlling a temperature at a top of the refining tower to be 20˜40° C. higher than that at a bottom of the reaction rectification tower, and side withdrawing the linear carbonate;

step 3: introducing the linear carbonate obtained in the step 2 into a melting crystallizer after passing through an adsorbent for adsorption, cooling to −50˜70° C. at a cooling rate of 1-3° C./h, and then keeping at this temperature for 1-5 h; and

step 4: purifying a crystal obtained in the step 3 by sweating at a controlled sweating temperature of 1-5° C. and a sweating ratio of 3-7% of a mass of the crystal, separating sweating liquor, and heating the remaining crystal to melting to obtain the linear carbonate.

In some embodiments of the present invention, in the step 1, the feeding molar ratio of the ethylene carbonate to the alcohol compound is 1:2˜1:10; and

preferably, the feeding molar ratio of the ethylene carbonate to the alcohol compound is 1:2˜1:6.

The alcohol compound is selected from at least one of methanol, ethanol, propanol and butanol.

Preferably, the alcohol compound is selected from two of methanol, ethanol, propanol and butanol.

In some embodiments of the present invention, the transesterification is conducted at a reaction pressure of 0.1˜0.25 MPa and a reaction temperature of 50˜120° C.

Preferably, the transesterification is conducted at a reaction pressure of 0.21 MPa and a reaction temperature of 110° C.

In some embodiments of the invention, the catalyst is selected from one or more of sodium methoxide, potassium carbonate, sodium carbonate, imidazole ionic liquid, quaternary ammonium ionic liquid and quaternary phosphonium ionic liquid.

In some embodiments of the invention, the adsorbent is selected from one or more of a molecular sieve, activated carbon and a cation exchange resin.

Compared with the prior art, the present invention has the following advantages:

The present invention provides a linear carbonate, wherein the hydroxyl concentration in the linear carbonate is no more than 100 ppm, and a free acid conversion rate of a solution with a concentration of 1 mol/L as formulated from the linear carbonate and lithium hexafluorophosphate is less than 1.2 after sealed storage under a condition of a constant temperature of 25° C. for 30 days. The present invention reduces an acidity conversion rate after lithium hexafluorophosphate is dissolved in the linear carbonate by controlling the hydroxyl concentration in the linear carbonate, so that when the linear carbonate is used as a solvent of an electrolyte solution, the energy density and discharge capacity of a battery can be improved, and further the safety performance and service life of the battery can be improved.

### DETAILED DESCRIPTION OF EMBODIMENTS

The following clearly and completely describes the technical solutions in the embodiments of the present invention with reference to the embodiments of the present invention. Apparently, the described embodiments are merely a part rather than all of the embodiments of the present invention. All other embodiments obtained by those of ordinary skills in the art based on the embodiments of the present invention without creative efforts shall fall within the claimed scope of the present invention.

The present invention will be described in detail through examples hereafter. The structural formulas of the compounds shown in Table 1 are all products that can be prepared by the preparation methods described in the examples of the present invention.

TABLE 1

| | |
|---|---|
| 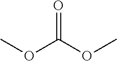 | Compound 1 |
| 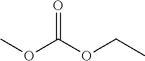 | Compound 2 |
| 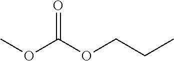 | Compound 3 |
| 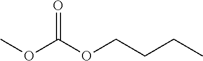 | Compound 4 |
| 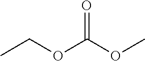 | Compound 5 |
| 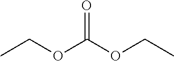 | Compound 6 |
| 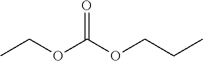 | Compound 7 |
| 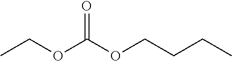 | Compound 8 |
| 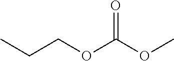 | Compound 9 |
| 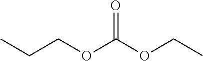 | Compound 10 |
| 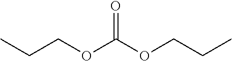 | Compound 11 |
| 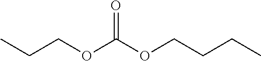 | Compound 12 |
| 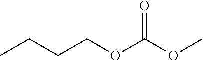 | Compound 13 |
| 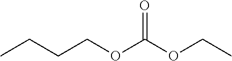 | Compound 14 |
| 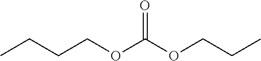 | Compound 15 |
| 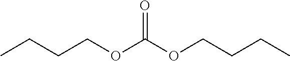 | Compound 16 |

### EXAMPLE 1

The preparation of a linear carbonate, including the following steps:

step 1: ethylene carbonate was mixed with potassium carbonate, and then introduced into a reaction rectification tower, and then methanol was introduced for transesterification at a reaction pressure of 0.15 MPa and a reaction temperature of 70° C., with the molar ratio of ethylene carbonate to methanol being 1:2;

step 2: a condensed fraction collected at a top of the reaction rectification tower was introduced into a refining tower, a temperature at a top of the refining tower was controlled to be 20° C. higher than that at a bottom of the reaction rectification tower, a light component was withdrawn from the top of the refining tower, a heavy component was withdrawn from the bottom of the refining tower, and dimethyl carbonate was withdrawn from the side line of the refining tower;

step 3: the dimethyl carbonate obtained in the step 2 was introduced into a melting crystallizer after adsorption by activated carbon, cooled to −50° C. at a cooling rate of 3° C./h, and kept at this temperature for 1 h; and

step 4: the dimethyl carbonate crystal obtained in the step 3 was heated to 1° C. for purification by sweating at a controlled sweating ratio of 3%, the sweating liquor was separated, and the remaining crystal was heated to melting to obtain electronic-grade dimethyl carbonate.

The tested hydroxyl solubility of the electronic-grade dimethyl carbonate was 80 ppm, and the tested acidity conversion rate of a formulated solution of lithium hexafluorophosphate in it was 1.15.

### EXAMPLE 2

The preparation of a linear carbonate, including the following steps:

step 1: ethylene carbonate was mixed with potassium carbonate, and then introduced into a reaction rectification tower, and then ethanol was introduced for transesterification at a reaction pressure of 0.2 MPa and a reaction temperature of 95° C., with the molar ratio of ethylene carbonate to ethanol being 1:3;

step 2: a condensed fraction collected at a top of the reaction rectification tower was introduced into a refining tower, a temperature at a top of the refining tower was controlled to be 40° C. higher than that at a bottom of the reaction rectification tower, a light component was withdrawn from the top of the refining tower, a heavy component was withdrawn from the bottom of the refining tower, and diethyl carbonate was withdrawn from the side line of the refining tower;

step 3: the diethyl carbonate obtained in the step 2 was introduced into a melting crystallizer after adsorption by activated carbon, cooled to 20° C. at a cooling rate of 1.5° C./h, and kept at this temperature for 3 h; and

step 4: the diethyl carbonate crystal obtained in the step 3 was heated to 4° C. for purification by sweating at a controlled sweating ratio of 5%, the sweating liquor was separated, and the remaining crystal was heated to melting to obtain electronic-grade diethyl carbonate. The testing results of the product were filled into Table 2.

### EXAMPLE 3

The preparation of a linear carbonate, including the following steps:

step 1: ethylene carbonate was mixed with sodium methoxide, and then introduced into a reaction rectification tower, and then methanol was introduced from an upper end of the rectification tower and ethanol was introduced from the lower end of the rectification tower for transesterification at a reaction pressure of 0.21 MPa and a reaction temperature of 110° C., with the molar ratio of ethylene carbonate to methanol and ethanol being 1:4:2;

step 2: a condensed fraction collected at a top of the reaction rectification tower was introduced into a refining tower, a temperature at a top of the refining tower was controlled to be 30° C. higher than that at a bottom of the reaction rectification tower, a light component was withdrawn from the top of the refining tower, a heavy component was withdrawn from the bottom of the refining tower, and methyl ethyl carbonate was withdrawn from the side line of the refining tower;

step 3: the methyl ethyl carbonate obtained in the step 2 was introduced into a melting crystallizer after adsorption by activated carbon, cooled to −10° C. at a cooling rate of 2° C./h, and kept at this temperature for 2 h; and

step 4: the methyl ethyl carbonate crystal obtained in the step 3 was heated to 2° C. for purification by sweating at a controlled sweating ratio of 4%, the sweating liquor was separated, and the remaining crystal was heated to melting to obtain electronic-grade methyl ethyl carbonate. The testing results of the product were filled into Table 2.

### COMPARATIVE EXAMPLE 1

The preparation of a linear carbonate, including the following steps:

step 1: ethylene carbonate was mixed with potassium carbonate, and then introduced into a reaction rectification tower, and then methanol was introduced for transesterification at a reaction pressure of 0.15 MPa and a reaction temperature of 70° C., with the molar ratio of ethylene carbonate to methanol being 1:2;

step 2: a condensed fraction collected at a top of the reaction rectification tower was introduced into a refining tower, a temperature at a top of the refining tower was controlled to be 45° C. higher than that at a bottom of the reaction rectification tower, a light component was withdrawn from the top of the refining tower, a heavy component was withdrawn from the bottom of the refining tower, and dimethyl carbonate was withdrawn from the side line of the refining tower;

step 3: the dimethyl carbonate obtained in the step 2 was introduced into a melting crystallizer after adsorption by activated carbon, cooled to −55° C. at a cooling rate of 4° C./h, and kept at this temperature for 1 h; and

step 4: the dimethyl carbonate crystal obtained in the step 3 was heated to 1° C. for purification by sweating at a controlled sweating ratio of 2%, the sweating liquor was separated, and the remaining crystal was heated to melting to obtain electronic-grade dimethyl carbonate. The testing results of the product were filled into Table 2.

### COMPARATIVE EXAMPLE 2

The preparation of a linear carbonate, including the following steps:

step 1: ethylene carbonate was mixed with sodium methoxide, and then introduced into a reaction rectification tower, and then methanol was introduced from an upper end of the rectification tower and ethanol was introduced from the lower end of the rectification tower for transesterification at a reaction pressure of 0.21 MPa and a reaction temperature of 110° C., with the molar ratio of ethylene carbonate to methanol and ethanol being 1:4:2;

step 2: a condensed fraction collected at a top of the reaction rectification tower was introduced into a refining tower, a temperature at a top of the refining tower was controlled to be 15° C. higher than that at a bottom of the reaction rectification tower, a light component was withdrawn from the top of the refining tower, a heavy component was withdrawn from the bottom of the refining tower, and methyl ethyl carbonate was withdrawn from the side line of the refining tower;

step 3: the methyl ethyl carbonate obtained in the step 2 was introduced into a melting crystallizer after adsorption by activated carbon, cooled to −10° C. at a cooling rate of 2° C./h, and kept at this temperature for 2 h; and

step 4: the methyl ethyl carbonate crystal obtained in the step 3 was heated to 6° C. for purification by sweating at a controlled sweating ratio of 8%, the sweating liquor was separated, and the remaining crystal was heated to melting to obtain electronic-grade methyl ethyl carbonate. The testing results of the product were filled into Table 2.

TABLE 2

| | | | Acidity |
|---|---|---|---|
| | | Hydroxyl | conversion |
| Group | Product | concentration | rate |
| Example 1 | Dimethyl carbonate | 80 ppm | 1.15 |
| Example 2 | Diethyl carbonate | 50 ppm | 1.13 |
| Example 3 | Methyl ethyl carbonate | 30 ppm | 1.08 |
| Comparative | Dimethyl carbonate | 130 ppm | 1.5 |
| Example 1 | | | |
| Comparative | Methyl ethyl carbonate | 120 ppm | 1.47 |
| Example 2 | | | |

The specific testing methods of the hydroxyl concentration in the linear carbonate obtained by the present invention and the acidity conversion rate of the linear carbonate were as follows:

1. Method of Testing Total Hydroxyl Concentration:

1.1 A hydroxyl-containing substance was identified in a compound 1 by a gas chromatograph-mass spectrometer, then the hydroxyl-containing substance in the compound 1 was quantified by gas chromatography and the moisture content in the compound 1 was tested by a moisture tester, and subsequently the total hydroxyl concentration was converted according to the concentration and moisture content of each hydroxyl substance.

1.2 The moisture content W (H₂O) in the compound 1 was tested in ppm according to the method in national standard GB/T 6284-2006;

1.3 If it was identified that the hydroxyl-containing substance in the compound was only a lower alcohol (with a carbon number≤4, an unknown concentration, recorded as W₀ in ppm), a spiked test can be carried out in the following manner.

m₀xg of the compound 1 was taken as the solvent, and then added with m₁yg of the lower alcohol standard substance (of analytically pure and above), so that m₁/m₀=0.05%, and a spiked sample of the linear carbonate was formulated at a concentration of the lower alcohol≈W₀+500 ppm.

Then, it was allowed that m₁/m₀=0.02%, 0.01%, 0.005% and 0.002% sequentially for formulation sequentially, so as to obtain spiked samples of the linear carbonate with concentrations of the lower alcohol of (W₀+200 ppm), (W₀+100 ppm), (W₀+50 ppm), and (W₀+20 ppm) respectively.

1.4 The gas chromatograph was set according to the following parameters, and then the aforementioned spiked samples of the carbonates were tested.

Model of chromatographic column: SE-54 capillary column

An injection port temperature was 200° C., an injection volume was 1 uL, a split ratio was 200:1, and a detector temperature was 250° C.

Programmed temperature increasing: the starting temperature was 100° C. and kept for 4 min, and then increased at 25° C./min to 250° C., and this temperature was kept for 10 min.

1.5 The peak area A of the lower alcohol was taken as a vertical coordinate and the additional spiked concentration (20, 50, 100, 200, 500 ppm) of each lower alcohol was taken as a horizontal coordinate, and linear fitting was conducted to obtain a curve with an equation of y=ax+b (wherein a and b>0), and thus the concentration of the lower alcohol in the compound 1 was W₀=b/a, in ppm.

1.6 If the compound 1 contained a variety of hydroxyl-containing substances such as A, B, C . . . etc., and the concentrations WA₀, WB₀, WC₀ . . . of A, B, C . . . etc. could be determined by the aforementioned methods, then the total hydroxyl concentration in the compound 1 was:

$$W ( OH . ppm ) = 2 W ( {H}_{2} O ) + {\sum }_{A}^{A , B , C…} \frac{17 * {x}_{A} * {W}_{A 0}}{{M}_{A}} = 2 W ( {H}_{2} O ) + \frac{17 * {x}_{A} * {W}_{A 0}}{{M}_{A}} + \frac{17 * {x}_{B} * {W}_{B 0}}{{M}_{B}} + \frac{17 * {x}_{C} * {W}_{C 0}}{{M}_{C}} +$$

Wherein M_{A}, M_{B}, M_{C} . . . were the relative molecular masses of A, B, C . . . etc., X_{A}, X_{B}, X_{C} . . . were the number of hydroxyl groups in the molecules A, B, C . . . etc. and W(H₂O) was the moisture content in the linear carbonate.

2. Method of Testing Acidity Conversion Rate f After Dissolution of Lithium Salt:

2.1 Dissolution conditions: the refined compound 1 (with the quality recorded as m₂) was taken, sealed and frozen below −10° C. for 1 h, then added with about 1/9 of its own mass of electronic-grade lithium hexafluorophosphate (with a mass recorded as m₃, and a free acid content less than 50 ppm) in a glove box (with a moisture content of less than 10 ppm, and an oxygen content of less than 20 ppm), and shaken well to obtain a solution of 10% lithium hexafluorophosphate dissolved in the linear carbonate, and the accurate concentration percentage of lithium hexafluorophosphate was recorded

$$x = \frac{{m}_{3}}{{m}_{2} + {m}_{3}} \times 100 \% .$$

2.2 Storage conditions: the formulated solution of the compound 1 in which 10% lithium hexafluorophosphate was dissolved, was transferred into a clean and dry aluminum flask and sealed. Then it was stored in a thermostat at 25° C. for 30 d. After 30 d, it was sampled in a glove box and determined for free acid.

2.3 Method of Testing Free Acid of Lithium Hexafluorophosphate and Lithium Hexafluorophosphate Solution:

2.3.1 Reagents: anhydrous acetonitrile (of analytically pure and above, and with a moisture content≤20 ppm), a methyl red indicator (0.1% Wt anhydrous acetonitrile), 0.01 mol/L of triethylamine-anhydrous acetonitrile titrant, triethylamine (of analytically pure and above), potassium hydrogen phthalate (standard reagent);

2.3.2 Formulation of Triethylamine-Anhydrous Acetonitrile Titrant and Standardization Method

2.3.2.1 1.01 g of triethylamine was weighed, diluted with anhydrous acetonitrile, transferred into a 1,000 mL volumetric flask, and brought to a constant volume with anhydrous acetonitrile to obtain a 0.01 mol/L triethylamine-anhydrous acetonitrile solution (formulated and used in a glove box);

2.3.2.2 A small amount of potassium hydrogen phthalate was taken as the standard reagent, and oven dried at 110° C. for 2 h for later use;

2.3.2.3 0.0204 g of potassium hydrogen phthalate (with its accurate mass recorded as m₄, in g, accurating to the 4th decimal place), dissolved by addition of ultrapure water, added dropwise with 3˜5 drops of a methyl red-anhydrous acetonitrile solution as the indicator, and titrated with a certain amount of the 0.01 mol/L triethylamine-anhydrous acetonitrile solution taken out of the glove box until the color of the solution changed from red to yellow, and the volume of the consumed triethylamine solution (recorded as V₁, in mL) was recorded. Parallel experiments were made with the requirement that the difference between two results was no more than 10% of the average of the two results;

2.3.2.4 Then the concentration of the triethylamine-anhydrous acetonitrile solution was calculated according to the following equation:

$$c = \frac{{m}_{4} \times 4.897}{{V}_{1}} , mol / L .$$

2.3.3 Method of Titrating Free Acid Content in Lithium Hexafluorophosphate and Lithium Hexafluorophosphate Solution

2.3.3.1 In a glove box, about 20 g of anhydrous acetonitrile was taken, added dropwise with 3˜5 drops of the indicator of 0.1% methyl red-anhydrous acetonitrile, added with the standardized triethylamine-anhydrous acetonitrile titrant until the indicator turned yellow (the addition amount was not recorded), then added with 10˜20 g of lithium hexafluorophosphate or a lithium hexafluorophosphate solution that had been stored for 30 d (with its accurate mass recorded as m₅, in g, accurating to the 2nd decimal place), titrated with the triethylamine-anhydrous acetonitrile solution until the solution just turned yellow again, and the volume of the consumed triethylamine-anhydrous acetonitrile titrant (V₂, in mL, accurating to the 2nd decimal place) was recorded. Parallel tests were made with the requirement that the difference between two determination results was no more than 20% of the average of the two results;

2.3.3.2 Then the free acid content in lithium hexafluorophosphate or the lithium hexafluorophosphate solution after storage was (in ppm, by HF):

$${W ( H F )}_{0} = \frac{20 \; 000 {cV}_{2}}{{m}_{5}} ;$$

The content of free acid in the lithium hexafluorophosphate solution after storage was (in ppm, by HF):

$${W ( H F )}_{1} = \frac{20000 {cV}_{2}^{′}}{{m}_{5}^{′}} ;$$

2.3.4 Calculation Formula of Acidity Conversion Rate:

$$f = \frac{{W ( H F )}_{1} - x * {W ( H F )}_{0}}{W ( OH ) \times ( 1 - x )} \times 100 \% .$$

For the linear carbonate of the present invention, by controlling the hydroxyl concentration in the linear carbonate, it can be ensured that after stored for a long period, the linear carbonate can have a small acidity conversion rate and more stable product properties after dissolution of a lithium salt in it.

The present invention has been further described by means of specific examples hereinabove, but it should be understood that the specific description here should not be construed as limiting the essence and scope of the present invention, and various modifications made by those of ordinary skills in the art to the aforementioned examples after reading the present specification are all within the claimed scope of the present invention.

1. A linear carbonate, comprising one or more of compounds of structural formula 1 below: 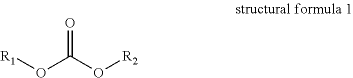
wherein R₁ and R₂ are respectively selected from one of alkyl groups containing 1˜4 carbon atoms; a hydroxyl concentration of the linear carbonate is no more than 100 ppm, and a free acid conversion rate of a solution with a concentration of 1 mol/L as formulated from the linear carbonate and lithium hexafluorophosphate is less than 1.2 after sealed storage under a condition of a constant temperature of 25° C. for 30 days.

2. The linear carbonate according to claim 1, wherein the compound of structural formula 1 is selected from one or more of the following compounds:

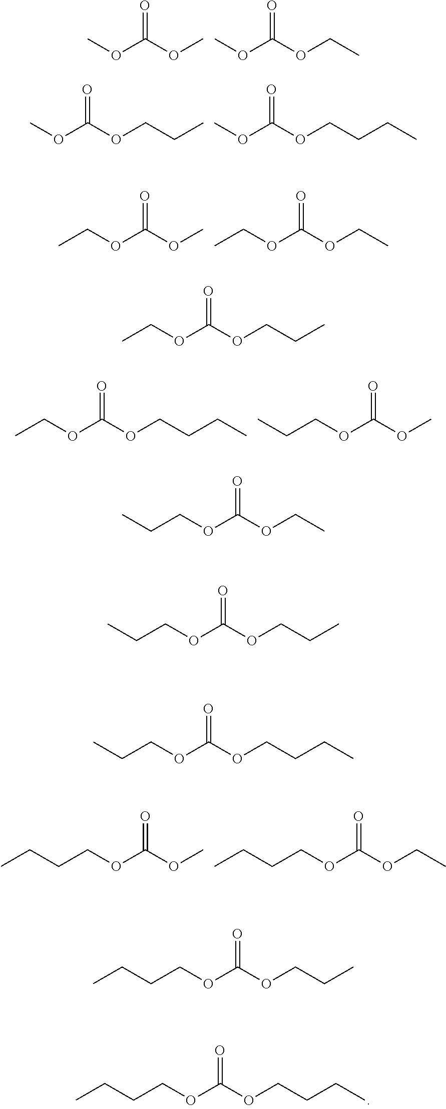

3. The linear carbonate according to claim 1, wherein the hydroxyl concentration of the linear carbonate is no more than 60 ppm.

4. A method for preparing the linear carbonate according to claim 1, comprising the following preparation steps:
step 1: mixing ethylene carbonate with a catalyst and then adding the mixture into a reaction rectification tower, and introducing an alcohol compound for transesterification;
step 2: introducing a condensed fraction collected at a top of the reaction rectification tower into a refining tower, controlling a temperature at a top of the refining tower to be 20˜40° C. higher than that at a bottom of the reaction rectification tower, and side withdrawing the linear carbonate;
step 3: introducing the linear carbonate obtained in the step 2 into a melting crystallizer after passing through an adsorbent for adsorption, cooling to (−50)-70° C. at a cooling rate of 1-3° C./h, and then keeping at this temperature for 1-5 h; and
step 4: purifying a crystal obtained in the step 3 by sweating at a controlled sweating temperature of 1-5° C. and a sweating ratio of 3-7% of a mass of the crystal, separating sweating liquor, and heating the remaining crystal to melting to obtain the linear carbonate.

5. The method for preparing the linear carbonate according to claim 4, wherein in the step 1, the feeding molar ratio of the ethylene carbonate to the alcohol compound is 1:2˜1:10; and
preferably, the alcohol compound is selected from at least one of methanol, ethanol, propanol and butanol.

6. The method for preparing the linear carbonate according to claim 4, wherein in the step 1, the transesterification is conducted at a reaction pressure of 0.1˜0.25 MPa and a reaction temperature of 50˜120° C.

7. The method for preparing the linear carbonate according to claim 4, wherein the catalyst is selected from one or more of sodium methoxide, potassium carbonate, sodium carbonate, imidazole ionic liquid, quaternary ammonium ionic liquid and quaternary phosphonium ionic liquid.

8. The method for preparing the linear carbonate according to claim 4, wherein the adsorbent is selected from one or more of a molecular sieve, activated carbon and a cation exchange resin.
